(19) United States
(12) Patent Application Publication
(10) Pub. No.: US 2019/0144908 A1
(43) Pub. Date: May 16, 2019

(54) HIGH CELL DENSITY ANAEROBIC FERMENTATION FOR PROTEIN EXPRESSION

(71) Applicant: White Dog Labs, Inc., New Castle, DE (US)

(72) Inventors: Bryan Patrick TRACY, Wilmington, DE (US); Shawn William JONES, Bear, DE (US); Aharon M. EYAL, Jerusalem (IL)

(73) Assignee: White Dog Labs, Inc., New Castle, DE (US)

(21) Appl. No.: 16/308,267

(22) PCT Filed: Jun. 9, 2017
(86) PCT No.: PCT/US2017/036768

§ 371 Date: Dec. 7, 2018

Related U.S. Application Data
(60) Provisional application No. 62/348,521, filed on Jun. 10, 2016.

(51) Int. Cl. C12P 21/00; C12N 15/74; C12N 1/20

(52) U.S. Cl. CPC C12P 21/00; C12N 1/20; C12N 15/74

(57) ABSTRACT

High cell density anaerobic fermentation for protein expression. A method for producing a protein is described, including providing a modified Butyribacterium methylotrophicum (Bme) strain host; providing a fermentation medium including a carbon source and a nitrogen source; culturing the modified host in the fermentation medium under anaerobic conditions, where the carbon source is metabolized and a fermentation broth including at least one protein is formed and optionally separating the protein; where the Bme cell density in the fermentation broth is greater than 15 g/L.

### CROSS-REFERENCE TO RELATED APPLICATION

The pending application claims the benefit of priority to U.S. Provisional Application No. 62/348,521 filed Jun. 10, 2016, the disclosure of which is expressly incorporated by reference in its entirety.

### FIELD OF THE INVENTION

The invention relates to the production of proteins using high cell density anaerobic fermentation.

### BACKGROUND

Protein (e.g., amylase and proteinase) expression is largely correlated with cell growth. Large quantities of ATP (adenosine triphosphate) are required for cell growth. Thus, protein expression hosts are grown in aerobic bioreactors whereby ATP is produced in significant quantities through oxidative respiration. However, aerobic fermentation requires a lot of gas compression and power input into the fermentation system to accomplish the necessary gas dispersion. Moreover, this is exacerbated by the desire to have very high cell density fermentations and short fermentation time periods.

Current common protein production, typically utilizes fungal species (e.g., Aspergillus niger, Aspergillus oryzae, Trichoderma reesei) and facultative anaerobes (e.g., Bacillus licheniformis, Bacillus clausii and Bacillus subtilis) to produce proteins at up to 75 g/L (grams/liter) titers in cultures with dry cell weights also as high as 75 g/L. Fungal fermentations commonly take 200-250 hours to reach maximum titers, and Bacillus fermentations take 40-80 hours.

There is a need for fermentation processes that produce protein at high titer, with low energy input, and in very short batch fermentation periods and/or by way of high cell density continuous fermentations. Anaerobic fermentation saves significantly on operating costs, reduces contamination risks, simplifies capital expenditures, and minimizes foaming. Short batch fermentations or high cell density continuous fermentations significantly increase fermentation volumetric productivity, and reduce capital expenditure costs.

### SUMMARY OF THE INVENTION

Provided is a method for producing a protein comprising (i) providing a modified Butyribacterium methylotrophicum (Bme) host; (ii) providing a fermentation medium comprising a carbon source and a nitrogen source; (iii) culturing said modified host in said fermentation medium under anaerobic conditions, whereby said carbon source is metabolized and a fermentation broth comprising at least one protein is formed; and (iv) optionally separating said at least one protein; wherein Bme cell density in said fermentation broth is greater than 15 g/L.

According to an embodiment, said providing a modified Bme host comprises modifying a naturally occurring Bme strain. According to an embodiment, said modifying results in increased expression of the at least one protein. According to an embodiment, said modifying comprises incorporating a transcriptional promoter operably linked to a nucleic acid sequence encoding the at least one protein into the host. According to an embodiment, said transcriptional promoter is constitutively active. According to an embodiment, said constitutively active transcriptional promoter comprises a thiolase (thl) promoter, a glyceraldehyde 3-phosphate dehydrogenase (gapDH) promoter, a phosphotransacetylase (pta) promoter, or a phosphate butyryltransferase (ptb) promoter. According to an alternative embodiment, said transcriptional promoter is inducible. According to an embodiment, said inducible transcriptional promoter comprises a lactose-inducible promoter or an arabinose-inducible promoter. According to an embodiment, said lactose-inducible promoter comprises a promoter for β-galactosidase selected from a bgaL promoter or a lacZ promoter. According to an embodiment, said arabinose-inducible promoter comprises a phosphoketolase (ptk) promoter.

According to an embodiment, said modifying comprises operably linking a ribosomal binding site to a nucleic acid sequence encoding the at least one protein. According to an embodiment, said modifying comprises transformation with a plasmid, modification of the host's genome, or a combination thereof. According to an embodiment, said modifying comprises introducing a plasmid having a copy number greater than 20 into the host.

According to an embodiment, said at least one protein is secreted. According to an embodiment, said modifying comprises modifying the protein secretion system of said host. According to an embodiment, said modifying comprises modifying a Sec secretion system. According to an embodiment, said modifying comprises modifying a twin arginine translocation (Tat) secretion system. According to an embodiment, said modifying comprises modifying an ATP-binding cassette (ABC) secretion system. According to an embodiment, said modifying comprises transformation with a plasmid, modification of the host's genome, or a combination thereof.

According to an embodiment, Bme host growth rate is greater than 0.7 g/L/hr (grams/Liter/hour). According to an embodiment, protein productivity of the at least one protein is greater than 0.01 g/L/hr. According to an embodiment, protein concentration of the at least one protein in said fermentation broth is greater than 1 g/L.

According to an embodiment, said protein is a recombinant protein. According to an embodiment, said at least one protein comprises an amylase, a protease, a xylanase, a lipase, interferon-α (hIFN-α), human interferon-β (hIFN-β), insulin-like growth factor 1 (IGF-1), human growth hormone (rHGH), and/or combinations thereof.

According to an embodiment, said culturing comprises continuous fermentation. According to an embodiment, said continuous fermentation comprises recycling one or more host cells.

According to an embodiment, the Bme host comprises a genetic modification in a gene of at least one secreted protease of said host. According to an embodiment, the genetic modification reduces express or secretion of the at least one secreted protease or deletes the at least one secreted protease from the genome of said host.

According to an embodiment, said carbon source comprises CO₂. According to an embodiment, said carbon source comprises a first feedstock and a second feedstock, wherein said first feedstock comprises a carbohydrate and wherein said second feedstock comprises CO, CO₂, carbonate, bicarbonate, H₂, glycerol, methanol, formate, urea or a combination thereof.

According to an embodiment, said method achieves greater production of the at least one protein than the combined amounts produced by heterotrophic and autotrophic fermentation with the same organism under the same conditions.

According to an embodiment, said metabolizing of the carbon source further produces acetic acid and the amount of acetic acid formed per biomass unit weight is less than about 50% of that formed in autotrophic fermentation with the same organism under the same conditions.

According to an embodiment, the carbon yield is at least 50%.

According to an embodiment, the ¹³C/¹²C isotope ratio of the carbon present in the at least one protein is less than that of atmospheric CO₂.

According to an embodiment, said fermentation medium comprises a steel mill produced CO composition.

According to an embodiment, said method comprises supplementing said fermentation medium with a mixture of CO₂ and hydrogen at a molar ratio in the range from 1:0.1 to 1:5. According to an embodiment, said method further comprises steam reforming of a hydrocarbon to form said mixture of CO₂ and hydrogen.

According to an embodiment, said carbon source comprises a carbohydrate selected from glucose and sucrose, and said modified Bme host metabolizes CO₂ produced during metabolizing said carbohydrate.

Also provided herein is a Butyribacterium methylotrophicum (Bme) cell comprising one or more genetic modifications for production of at least one protein, which modified cell is characterized by (a) an ability to reach, in a medium comprising a carbon source and a nitrogen source, cell density greater than 15 g/L; (b) a cell growth rate greater than 0.7 g/L/hr, and (c) production of said at least one protein in an anaerobic environment.

According to an embodiment, the Bme cell is capable of achieving protein productivity of the at least one protein of greater than 0.01 g/L/hr.

According to an embodiment, the Bme cell comprises a heterologous transcriptional promoter operably linked to a nucleic acid sequence encoding the at least one protein. In an embodiment, the transcriptional promoter is constitutively active. According to an embodiment, the constitutively active transcriptional promoter comprises a thiolase (thl) promoter, a glyceraldehyde 3-phosphate dehydrogenase (gapDH) promoter, a phosphotransacetylase (pta) promoter, or a phosphate butyryltransferase (ptb) promoter. According to an embodiment, said transcriptional promoter is inducible. According to an embodiment, the inducible transcriptional promoter comprises a lactose-inducible promoter or an arabinose-inducible promoter. According to an embodiment, said lactose-inducible promoter comprises a promoter for β-galactosidase selected from a bgaL promoter or a lacZ promoter. According to an embodiment, the arabinose-inducible promoter comprises a phosphoketolase (ptk) promoter.

According to an embodiment, the Bme cell comprises a ribosomal binding site operably linked to a nucleic acid sequence encoding the at least one protein.

According to an embodiment, the Bme cell is modified by transformation with a plasmid, modification of the cell's genome, or a combination thereof.

According to an embodiment, the Bme cell is modified by introducing a plasmid having a copy number greater than 20 into the cell.

According to an embodiment, the Bme cell comprises a modified protein secretion system. According to an embodiment, a Sec secretion system is modified. According to an embodiment, a twin arginine translocation (Tat) secretion system is modified. According to an embodiment, an ATP-binding cassette (ABC) secretion system is modified.

According to an embodiment, the Bme cell may be modified by transformation with a plasmid, modification of the cell's genome, or a combination thereof.

According to an embodiment, the Bme cell comprises one or more genetic modifications for production of at least one exogenous protein.

According to an embodiment, the Bme cell comprises one or more genetic modifications for increased production of at least one endogenous protein.

According to an embodiment, the Bme cell comprises a genetic modification in a gene of at least one secreted protease. According to an embodiment, the Bme cell comprises a genetic modification that reduces expression or secretion of the at least one secreted protease or that deletes the at least one secreted protease from the genome.

Also provided herein is a protein preparation comprising at least one protein and a modified Bme cell as described above. According to an embodiment, said at least one protein is a recombinant protein. According to an embodiment, said at least one protein comprises an amylase, a protease, a xylanase, a lipase, interferon-α (hIFN-α), human interferon-β (hIFN-β), insulin-like growth factor 1 (IGF-1), human growth hormone (rHGH), and/or combinations thereof. According to an embodiment, the ¹³C/¹²C isotope ratio of the carbon present in said at least one protein is less than that of atmospheric CO₂.

### DETAILED DESCRIPTION OF THE INVENTION

### Definitions

As used herein the term “carbon yield” refers to carbon yield based on the total amount of carbon in produced products, specifically protein, metabolites, and biomass, divided by the total amount of carbon metabolized from a provided carbon source.

As used herein the term “constitutively active promoter” refers to a promoter that is active in the presence or in the absence of an external stimulus.

As used herein the term “inducible promoter” refers to a promoter that is active when turned on by an external stimulus.

As used herein, Bme refers to Butyribacterium methylotrophicum.

As used herein the term “protein productivity” refers to the volumetric productivity of the at least one protein of interest from the Bme cell.

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

Provided is a method for producing a protein comprising (i) providing a modified Butyribacterium methylotrophicum (Bme) strain host; (ii) providing a fermentation medium comprising a carbon source and a nitrogen source; (iii) culturing said modified host in said fermentation medium under anaerobic conditions, whereby said carbon source is metabolized and a fermentation broth comprising at least one protein is formed; and (iv) optionally separating said at least one protein; wherein Bme cell density in said fermentation broth is greater than 15 g/L.

According to an embodiment, said carbon source may be selected from the group consisting of carbohydrates, CO, CO₂, carbonate, bicarbonate, H₂, glycerol, methanol, formate, urea and a combination thereof.

According to an embodiment, said nitrogen source may be selected from the group consisting salts of ammonia, e g ammonium acetate, ammonium bicarbonate, ammonium chloride, ammonium nitrate, ammonium sulfate and diammonium phosphate, complex nitrogen sources, e.g. yeast extract, corn water-insolubles, stillage, molasses and urea and a combination thereof.

According to an embodiment, the host strain may be any member of the genus Butyribacterium. According to an embodiment, said providing a modified Bme strain host comprises modifying a Bme strain. According to an embodiment, said providing a modified Bme strain comprises modifying a native Bme strain. According to an embodiment, said providing a modified Bme strain comprises further modifying an already genetically modified Bme strain host. According to an embodiment, said modifying is directed to increased protein expression. According to an embodiment, expression of one or more proteins by said modified Bme strain host may be greater than such protein expression by a corresponding native Bme strain by at least 10%, by at least 20%, by at least 50%, or by at least 100%.

According to an embodiment, said modifying comprises operably linking a transcriptional promoter to a DNA sequence encoding a protein to be produced. The promoter may be heterologous to the host or plasmid. In an alternative embodiment the promoter may be endogenous to the host or plasmid. According to an embodiment, said transcriptional promoter promotes production of mRNA transcripts, wherein said transcripts rank within the top 20% of all transcripts within a cell, top 15% of all transcripts, top 10% of all transcripts, or top 5% of all transcripts.

According to an embodiment, said transcriptional promoter may be constitutively active. According to an alternative embodiment, said transcriptional promoter may be inducible. According to an embodiment, said transcriptional promoter may be physically regulated, e.g. activated by physical parameters like light or different temperatures. According to an alternative embodiment, said transcriptional promoter may be chemically-regulated, i.e. activated by a chemical compound ranging from small metal ions to sugars to large molecules like steroids. According to an embodiment, said chemical compound is selected from isopropyl β-D-1-thiogalactopyranoside (IPTG), D-arabinose and anhydrotetracycline.

According to an embodiment, said modifying comprises operably linking a ribosomal binding site to a nucleic acid sequence encoding the at least one protein. The ribosomal binding site (RBS), which in an embodiment may include the Shine-Dalgarno sequence, recruits the ribosome to initiate protein translation. The RBS used may be optimized for protein expression in Bme.

According to an embodiment, said modifying is plasmid-based, chromosomal-based, or a combination thereof. For example, the modifying may comprise transformation with a plasmid, modification of the host's genome, or a combination thereof. Modifications can be introduced either on a plasmid or vector, incorporated into the chromosome, or by way of a combination of chromosomal integration and plasmid introduction.

According to an embodiment, said modifying comprises introducing a plasmid having a copy number greater than 100 into the host. The copy number of a plasmid in a given host is highly dependent upon the episome used to replicate the plasmid. An episome that results in a high-copy number plasmid, or a relaxed plasmid, may be used in the modified Bme strain. The copy number may be greater than 20, greater than 50, greater than 100, or greater than 200.

According to an embodiment, said protein may be at least partially secreted, at least 60% secreted, at least 70%, at least 80% or at least 90%.

According to an embodiment, said modifying may comprise modifying the protein secretion system of said Bme strain. According to an embodiment, said modifying comprises modifying the Sec secretion system.

According to an embodiment, said modifying comprises modifying the twin arginine translocation (Tat) secretion system. According to an embodiment, said modifying comprises modifying the ATP-binding cassette (ABC) secretion system. According to an embodiment, said modifying is plasmid-based, chromosomal-based, or a combination thereof. Modifications can be introduced either on a plasmid, incorporated into the chromosome of the host, or be achieved by a combination of chromosomal integration and plasmid introduction.

General Secretion (Sec) System

To accomplish translocation of the protein across the cytoplasmic membrane, a Sec system (Sec) comprising the following three subunits may be utilized: a protein targeting component, a motor protein, and a membrane integrated conducting channel. Such a system may be referred to as SecYEG. Additional accessory proteins may also utilized for proper function of the system. Proteins secreted by the Sec system are usually in their unfolded state. Proteins are targeted for secretion by a hydrophobic signal sequence at the N-terminus. This signal sequence is typically 20 amino acids long and contain 3 regions: a positively charged amino terminal, a hydrophobic core, and a polar carboxyl-terminal. For many proteins targeted for extracellular secretion, this signal sequence is specific for the SecB protein. SecB binds to the unfolded protein and prevents proper folding. SecB delivers the unfolded protein to SecA, which guides the protein into the SecYEG channel and serves as the ATPase that provides the energy for translocation. Once the protein is released from SecA, a protease cleaves off the SecB signal sequence from the unfolded protein, and the protein is then translocated and folded after secretion. Secretion can also be accomplished using a specialized SecA2 protein.

Twin Arginine Translocation (Tat) Secretion System

Proteins secreted by the Tat system are generally folded, unlike those translocated by the Sec system. Secretion is accomplished by two proteins, TatA and TatC, which form one multi-functional protein complex. The signal sequence for the Tat system comprises a pair of two arginines at the N-terminus of the folded protein (typically, the signal sequence is serine-arginine-arginine). The Tat system does not appear to require ATP but is dependent upon a proton motive force across the membrane.

ATP-Binding Cassette (ABC) Secretion System

ABC-systems typically target specific molecules, such as nutrients for importation into the cell or toxic compounds for exportation out of the cell. In general, the system consists of two hydrophobic membrane-spanning domains and two hydrophilic domains carrying the associated ABC domain. Importation or exportation is achieved by coupling the energy released from the hydrolysis of ATP with the translocation of molecules or proteins across a membrane. Because of the small size of the ABC channel, proteins are typically secreted unfolded, similar to the Sec system. There is no universal peptide signal for the ABC system, as they are specialized for specific proteins or molecules.

Proteases

Proteases are a diverse group of enzymes whose primary function is to perform proteolysis (i.e., the hydrolysis of peptide bonds). Often times, proteolysis results in the destruction and degradation of full-length proteins. Unfolded or improperly folded proteins are more susceptible to proteases than properly folded proteins. Proteases serve an important function intracellularly by degrading unnecessary proteins or potentially harmful proteins. Proteases can also be secreted by the cell to degrade extracellular proteins. This can pose a significant challenge if the goal is extracellular secretion of a target protein, especially if the protein is secreted in the unfolded state (e.g., by the Sec system).

Proteases can be dividing in a number of different categories. One classification is by the optimal pH of the protease, and this category of proteases can be divided into acid, neutral, or basic (a.k.a. alkaline) proteases. Another classification system uses the catalytic residue as the basis for classification, and this category of proteases can be divided into serine proteases, cysteine proteases, threonine proteases, aspartic proteases, glutamic proteases, metalloproteases, and asparagine peptide lysases. There are also oligopeptidases that target shorter oligopeptides rather than full-length proteins.

A number of these types of enzymes have been identified as being secreted, including metalloproteases, serine proteases, neutral proteases, and alkaline proteases. Deletion of these proteases improves the yield of extracellular protein, though their deletion may in certain circumstances also increase cell lysis and reduce growth rate.

According to an embodiment, Bme cell density in said fermentation broth may be greater than 15 g/L, greater than 20 g/l, greater than 30 g/l, greater than 40 g/l, or greater than 50 g/l.

According to an embodiment, Bme cell growth rate may be greater than 0.7 g/L/hr, greater than 1.0 g/L/hr, greater than 2 g/L/hr, greater than 5 g/L/hr, or greater than 10 g/L/hr.

According to an embodiment, protein productivity may be greater than 0.01 g/L/hr, greater than 0.05 g/L/hr, greater than 0.1 g/L/hr, greater than 0.5 g/L/hr, or greater than 1.0 g/L/hr.

According to an embodiment, protein concentration in said fermentation broth may be greater than 1 g/L, greater than 5 g/L, greater than 10 g/L, greater than 20 g/L or greater than 50 g/L.

Host cell density, host cell growth rate, protein productivity and/or protein concentration as described above may be maintained throughout the fermentation process or for only a part of the fermentation process. The cell density/growth rate/protein productivity/protein concentration may be achieved at a certain point after fermentation has begun or may be achieved at the beginning or by the end of the fermentation process or batch.

According to an embodiment, said protein is a recombinant protein. According to an embodiment, said protein is a heterologous protein. According to an embodiment, said protein is selected from the group consisting of amylases, proteases, xylanases, and lipases. The pharmaceutical proteins include human interferon-α (hIFN-α), human interferon-β (hIFN-β), insulin-like growth factor 1 (IGF-1), and human growth hormone (rHGH) and combinations thereof.

According to an embodiment, said fermentation may be conducted under anaerobic conditions with a dissolved oxygen concentration of less than 1 mg/L, less than 0.8 mg/L, less than 0.6 mg/L, less than 0.4 mg/L, or less than 0.2 mg/L. According to an embodiment, said fermentation may be conducted at a temperature in the range between 28° C. and 37° C. According to an embodiment, fermentation pH may be in the range between 4.5 and 6.5.

According to an embodiment, said culturing comprises continuous fermentation. According to an embodiment, said continuous fermentation comprises recycling one or more host cells. According to an embodiment, said cell recycling comprises membrane filtration.

According to an embodiment, said carbon source comprises CO₂. According to an embodiment, said carbon source comprises a first feedstock and a second feedstock, wherein said first feedstock comprises a carbohydrate and wherein said second feedstock comprises CO, CO₂, carbonate, bicarbonate, H₂, glycerol, methanol, formate, urea or a combination thereof.

According to an embodiment, said fermentation medium comprises a steel mill produced CO composition.

According to an embodiment, the method further comprises supplementing said fermentation medium with a mixture of CO₂ and hydrogen at a molar ratio in the range from 1:0.1 to 1:5. According to an embodiment, the method further comprises steam reforming of a hydrocarbon to form said mixture of CO₂ and hydrogen.

According to an embodiment, the first feedstock comprises a carbohydrate selected from glucose and sucrose, and said modified Bme metabolizes CO₂ produced during metabolizing said carbohydrate.

According to an embodiment, said method achieves greater production of the at least one protein than the combined amounts produced by heterotrophic and autotrophic fermentation with the same organism under the same conditions, at least 5% greater, at least 10% or at least 20% greater.

According to an embodiment, said metabolizing further produces acetic acid and the amount of acetic acid formed per biomass unit weight is less than about 50% of that formed in autotrophic fermentation with the same organism under the same conditions, less than 40%, less than 30%, less than 20%, or less than 10%.

According to an embodiment, the carbon yield, based on the total amount of carbon in produced protein divided by the total amount of carbon metabolized from said first carbon source is at least 50%, at least 60%, at least 70%, at least 80%, at least 90%, at least 100%, at least 110%, at least 120%, at least 130%, at least 140%, at least 150%, or at least 160%.

According to an embodiment, the ¹³C/¹²C isotope ratio of the carbon present in said protein is less than that of atmospheric CO₂.

According to an embodiment, the method comprises separating protein from said fermentation broth. According to an embodiment, the method comprises separating secreted protein from said fermentation broth. According to an embodiment, said separating comprises at least one of precipitation and ultrafiltration.

### EXAMPLES

### Example 1

A modified Bme strain is cultured under anaerobic conditions in a fermentation medium comprising glucose as the carbon source and yeast extract and ammonium sulfate as the nitrogen sources. The pH is 6.5 and the temperature is 35° C. Cell densities after 17 hours and 23 hours are about 10 g/L and about 15 g/L, respectively. Glucose consumption is about 40 g/L.

### Example 2

In order to produce a protein of interest in Butyribacterium methylotrophicum (Bme), the Bme strain is modified with a plasmid, which is introduced to Bme using an electroporation method. This plasmid contains an expression cassette containing a transcriptional promoter, a ribosomal-binding site (RBS), the coding sequence of the protein of interest, and a transcriptional terminator. All of these elements, except for the terminator, can be optimized to enhance protein expression.

For the transcriptional promoter, either a constitutively active, strong promoter, meaning the promoter is always active and produces a high amount of mRNA, or an inducible promoter, meaning the promoter is inactive until an inducer molecule is added to the culture, can be used. An inducible promoter has the advantage of not stressing the cell with protein production until desired. The strong promoter could be a thiolase (thl) promoter, a glyceraldehyde 3-phosphate dehydrogenase (gapDH) promoter, a phosphotransacetylase (pta) promoter, or a phosphate butyryltransferase (ptb) promoter selected from either Bme or another organism. The inducible promoter could be a lactose-inducible β-galactosidase promoter, like a bgaL or lacZ promoter, or a phosphoketolase arabinose-inducible promoter.

Protein translation can also be improved. The RBS is optimized to improve protein translation and thus produce more protein compared to a wild-type (WT) RBS. Optimization includes the binding site itself, typically AGGAGG, though different variations can be more effective. The spacing or sequence between the RBS and the start codon (typically ATG) may also be optimized. Additionally, the coding sequence of the protein of interest is modified such that rare codons, like CGA, AGG, and CTA, are replaced by more preferred codons to improve protein translation and thus produce more protein compared to the WT sequence.

Finally, the plasmid backbone itself is modified to use a replication region that maintains the plasmid within Bme at a copy number (i.e., number of copies of plasmid within the cell) greater than 20.

The modified strain of Bme is cultured in ATCC 1136 medium containing 80 g/L of glucose, 0.3 g/L KH₂PO₄, 0.3 g/L (NH₄)₂SO₄, 0.6 g/L NaCl, 0.12 MgSO₄.7H₂O, 0.08 g/L CaCl₂.2H₂O, 0.2 g/L K₂HPO₄.3H₂O, 0.5 g/L cysteine-HCl, 1 g/L yeast extract, 10 mL/L Wolfe's Mineral solution, and 10 mL/L Wolfe's Vitamin solution. Culture is grown at 35° C. at a pH of 6.5 and under anaerobic conditions. If an inducible promoter is used, once the culture reaches mid-exponential phase (OD 1-5), or other phase as determined by the operator, an inducer molecule, like isopropyl β-D-1-thiogalactopyranoside (IPTG) for a lactose-inducible promoter or arabinose for an arabinose-inducible promoter, is added at a specific concentration to maximize the expression of the promoter. Growth continues along with protein expression.

All glucose is consumed within 23 hrs and the cell mass reaches over 15 g/L. The growth rate of the modified Bme is greater than 0.7 g/L/hr with the production rate of the protein of interest reaching at least 0.01 g/L/hr. The protein achieves a concentration of at least 1 g/L. The carbon yield at the end of fermentation is at least 50%, calculated as the ratio of total carbon in products (including biomass, metabolites, and the protein of interest) to total sugar carbon consumed (i.e., glucose).

### Example 3

In order to improve the secretion of the protein of interest from Butyribacterium methylotrophicum (Bme), its protein secretion system is modified. The primary system for most protein secretion is the Sec system. This system consists of the SecY, SecE, and SecG transmembrane channel and SecA, which binds to the protein of interest and brings it to the SecYEG channel. The accessory proteins SecD and SecF assist the overall machinery under high expression of proteins. There is also a signal recognition particle (SRP) protein that binds to the signal recognition sequence of the forming peptide chain along with the helper protein Ffh. The SRP/Ffh complex is then recognized by FtsY, which then helps target and guide the unfolded protein to the transmembrane channel. All these genes are present in Bme and are overexpressed to increase protein secretion of the protein of interest. Overexpression can be achieved by either plasmid expression, similar to that described in Example 2, or by chromosomal manipulation. For chromosomal manipulation, the transcriptional promoter and RBS of the WT genes is replaced by a promoter or RBS that results in a higher amount of protein, compared to the WT promoter or RBS, similar to that described in Example 2. Alternatively, additional copies of the genes are also introduced to the chromosome, again resulting in an increased amount of protein.

To accomplish secretion, a signal peptide must be attached to the protein of interest, if it does not already have one. The signal peptide is on the N-terminus of the protein of interest and consists of a positively charged amino terminal, a hydrophobic core, and a polar carboxyl-terminal. This sequence may be optimized in order to maximize protein secretion. A good signal peptide used frequently is that for subtilisin from Bacillus subtilis.

The optimized coding sequence from Example 2 must be modified further to include the optimized signal peptide. That plasmid is then transformed into the modified Bme strain with the enhanced Sec machinery and grown under the conditions specified in Example 2 to produce the protein to interest and secrete the protein.

### Example 4

Bacteria also have a Sec-independent secretion system called the twin-arginine transport (Tat) system. Unlike Sec-translocation, the Tat system can translocate folded proteins, which can be a significant advantage for difficult to fold recombinant proteins. Since a native Tat system has not be identified in Butyribacterium methylotrophicum (Bme), a recombinant one is expressed. Bacillus subtilis (Bsu) has two well-characterized Tat systems consisting of two proteins each: TatAyCy and TatAdCd. Two expression cassettes are constructed of the two pairs of genes (i.e., tatAyCy and tatAdCd) to optimize expression of them, as in Example 2. These cassettes are expressed on a plasmid or are integrated into the genome of Bme, similar to Example 3.

Like Example 3 as well, the signal peptide for Tat recognition must also be attached to the N-terminus of the protein of interest. The recognition signal is typically R-R-X-#-#, where R is arginine, X is any amino acid, and # is a hydrophobic amino acid (i.e., glycine, alanine, leucine, isoleucine, proline, phenylalanine, methionine, or tryptophan). This signal sequence is attached to the protein of interest to target it for secretion. In order to maximize protein secretion, this signal sequence may be optimized, and various combinations may be tested until an optimal one is found. A good starting signal sequence is that for PhoD from Bsu.

The optimized expression cassette for the protein of interest, containing the optimized signal peptide sequence, is put into a plasmid, as described in Example 2, and then transformed into the modified Bme strain containing the recombinant Tat system. The strain is then grown as described in Example 2 to produce and secrete the protein of interest.

### Example 5

Bacteria naturally secrete a number of enzymes, including proteases, which are enzymes that hydrolyze and degrade other proteins. Proteases serve an important function by degrading improperly folded proteins and other extracellular proteins. However, these same proteases may degrade the protein of interest under certain circumstances, especially if the protein of interest is secreted by the Sec system, since such a protein is secreted in the unfolded state and more vulnerable to proteases. Therefore to further improve Butyribacterium methylotrophicum (Bme) protein production, these secreted proteases are deleted from the genome.

First, all proteases are identified within the genome. Then, the N-terminus region is analyzed to detect a Sec signal peptide. Proteases with a signal peptide are targeted for deletion. A number of different classes of proteases are targeted including metalloproteases, serine proteases, neutral proteases, and alkaline proteases. Deletion is accomplished using one of several techniques, such as CRISPR-Cas or homologous recombination. A plasmid expressing a protein of interest, as developed in Example 2, is then transformed into the deletion strains, and total secreted protein is compared to the WT Bme strain. If a protease cannot be deleted, as some may be essential for growth, its expression is reduced using CRISPR interference or anti-sense RNA targeted against the protease's mRNA sequence.

### Example 6

Protein production is an energy intensive process requiring significant amounts of ATP. Most aerobic species generate this energy by oxidative phosphorylation. Anaerobic organisms though must utilize other pathways to generate the energy needed. Butyribacterium methylotrophicum (Bme) generates ATP through two pathways: glycolysis and membrane-bound ATPases. In glycolysis, sugars (such as glucose) are broken down and energy is generated, both ATP and NADH. For the membrane-bound ATPases, their action is driven by an ion pump associated with a proton gradient generated by an Rnf complex. Membrane-bound ATPases are essential for a class of bacteria called acetogens that use the Wood-Ljungdahl pathway to fix Cl substrates (like CO₂, CO, or methanol). This is because under autotrophic conditions (the absence of sugar) when CO₂ is fixed into acetate, no net ATP is produced. Net ATP production is only accomplished by the membrane-bound ATPases. These same ATPases also function under non-autotrophic conditions to further increase the pool of ATP for Bme. Thus under high ATP demand, such as protein production, cultures of Bme are fed both glucose and a reducing source (such as H₂, CO, methanol, etc.) to help drive the proton gradient powering the ATPase and thus generate more ATP.

Strains generated in Examples 2-5 are grown on glucose while also fed H₂, CO, methanol, or glycerol. The strains are able to further increase total cell mass and thus total protein productivity.

The scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for producing a protein comprising
(i) providing a modified Butyribacterium methylotrophicum (Bme) host;
(ii) providing a fermentation medium comprising a carbon source and a nitrogen source;
(iii) culturing said modified host in said fermentation medium under anaerobic conditions, whereby said carbon source is metabolized and a fermentation broth comprising at least one protein is formed; and
(iv) optionally separating said at least one protein;
wherein Bme cell density in said fermentation broth is greater than 15 g/L.

2. The method according to claim 1, wherein said providing a modified Bme host comprises modifying a naturally occurring Bme strain.

3. The method according to claim 2, wherein said modifying results in increased expression of the at least one protein.

4.-11. (canceled)

12. The method according to claim 2, wherein said modifying comprises transformation with a plasmid, modification of the host's genome, or a combination thereof.

13. (canceled)

14. The method according to claim 1, wherein said at least one protein is secreted.

15. The method according to claim 2, wherein said modifying comprises modifying the protein secretion system of said host.

16.-19. (canceled)

20. The method according to claim 1, wherein said Bme host has a growth rate that is greater than 0.7 g/L/hr.

21. The method according to claim 1, wherein protein productivity of the at least one protein is greater than 0.01 g/L/hr.

22. The method according to claim 1, wherein protein concentration of the at least one protein in said fermentation broth is greater than 1 g/L.

23. The method according to claim 1, wherein said protein is a recombinant protein.

24.-26. (canceled)

27. The method according to claim 2, wherein the Bme host comprises a genetic modification in a gene of at least one secreted protease of said host.

28. The method according to claim 27, wherein the genetic modification reduces expression or secretion of the at least one secreted protease or deletes the at least one secreted protease from the genome of said host.

29. (canceled)

30. The method according to claim 1, wherein said carbon source comprises a first feedstock and a second feedstock, wherein said first feedstock comprises a carbohydrate and wherein said second feedstock comprises CO, CO₂, carbonate, bicarbonate, H₂, glycerol, methanol, formate, urea or a combination thereof.

31.-38. (canceled)

39. A Butyribacterium methylotrophicum (Bme) cell comprising one or more genetic modifications for production of at least one protein, which modified cell is characterized by (a) an ability to reach, in a medium comprising a carbon source and a nitrogen source, cell density greater than 15 g/L; (b) a cell growth rate greater than 0.7 g/L/hr, and (c) production of said at least one protein in an anaerobic environment.

40. The Bme cell according to claim 39, which is capable of achieving protein productivity of the at least one protein of greater than 0.01 g/L/hr.

41.-48. (canceled)

49. The Bme cell according to claim 39, which has been modified by transformation with a plasmid, modification of the cell's genome, or a combination thereof.

50. (canceled)

51. The Bme cell according to claim 39, comprising a modified protein secretion system.

52.-57. (canceled)

58. The Bme cell according to claim 39, comprising a genetic modification in a gene of at least one secreted protease.

59. The Bme cell according to claim 58, comprising a genetic modification that reduces expression or secretion of the at least one secreted protease or that deletes the at least one secreted protease from the genome.

60. A protein preparation comprising at least one protein and a modified Bme cell according to claim 39.

61. The protein preparation according to claim 60, wherein said at least one protein is a recombinant protein.

62. (canceled)

63. (canceled)
